(19) United States
(12) Patent Application Publication
(10) Pub. No.: US 2002/0068362 A1
(43) Pub. Date: Jun. 6, 2002

(54) Increased transgene expression in retroviral vectors having a scaffold attachment region

(76) Inventors: Lesley Jean Murray, San Jose, CA (US); Ivan Plavec, Sunnyvale, CA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/725,433

(22) Filed: Feb. 6, 2001

Related U.S. Application Data
(60) Provisional application No. 60/168,193, filed on Nov. 30, 1999.

(51) Int. Cl. C12N 15/867; C12N 7/00

(52) U.S. Cl. 435/456; 435/320.1; 435/235.1

(57) ABSTRACT

Transcriptional silencing of transgene expression from Moloney murine leukemia (MoMLV) retroviral vectors has been a hurdle in bringing effective gene therapy to the clinic. The present invention used an optimized transduction protocol for human hematopoietic stem cells (HSC) from mobilized peripheral blood (MPB) to compare MoMLV and mouse stem cell virus (MSCV) vectors, with or without addition of a scaffold attachment region (SAR) from the human interferon-β gene. To estimate retroviral vector supernatant quality, transgene delivery to CD34⁺ cells was quantitated 72 hours after transduction using real-time PCR. To estimate the impact of vector backbone and SAR on transgene expression, the percentage of HSC progeny expressing retroviral transgene was compared 72 hours after transduction, and following 5 week stromal culture, or 6-8 week in vivo HSC repopulation assays (SCID-hu bone and NOD/SCID). The predominant effect of SAR, observed following long term assays, was to increase the mean fluorescence intensity (MFI) of transgene expression among HSC progeny in both in vivo bone repopulation models (3-4 fold), and 2 fold following long term stromal cultures. Using MSCV-SAR vector and the optimized transduction protocol, transgene expression was observed among a mean of 10% of donor HSC progeny in the SCID-hu bone (range 0.6-43%), and among 3-5% of human HSC progeny in bone marrow and peripheral blood of NOD/SCID mice.

This application claims the benefit under 35 U.S.C., 119(e) of U.S. Provisional Application No. 60/168,193, filed Nov. 30, 1999, for “Increased Transgene Expression in Retroviral Vectors Having Scaffold Attachment Region,” the disclosure of which is hereby incorporated by reference in its entirety.

This invention relates to new retroviral vectors having scaffold attachment region, as well as packaging cell lines for producing such vectors, and methods for increasing expression of a transgene and for therapeutically administering such retroviruses.

### BACKGROUND OF THE INVENTION

Clinical therapy of HIV infection using retrovirally gene transduced HSC is an important goal, because integration of transduced anti-HIV genes in pluripotent HSC may allow long-term or even life-long expression among both myeloid and T cell lineages. To achieve sustained high level expression of a therapeutic transgene in patients, it will be necessary to increase the efficiency of gene delivery by retroviral vectors to long term repopulating pluripotent HSC.

Murray et al, 1999, Young et al., 1999, Moritz et al., and 1994, Hanenberg et al., 1997, recently described optimization of HSC transduction conditions, culturing with thrombopoietin (TPO), flt3 and kit ligands (TFK) on plates coated with the CH-296 fragment of fibronectin (RetroNectin™, RN), which achieved 88% gene marking of primitive long-term culture-derived colony-forming cells (LTC-CFC).

However, only about 10% of CD34⁺ cells following stromal culture expressed transgene, indicating block or shutdown of gene expression (Murray et al., 1999b). The MoMLV vectors currently in use (Miller and Rosman, 1989) are subject to position-dependent variation in gene expression, and transcriptional silencing. This may be due to de novo methylation of the 5′ MoMLV LTR in HSC (Challita et al., 1994, 1995) and negative regulatory transcription factors that bind to the LTR and the primer binding site (Flanagan et al., 1989, Petersen et al., 1991).

### SUMMARY OF THE INVENTION

In one embodiment, the present invention relates to a retroviral vector comprising at least one transgene operatively linked to a promoter, the promoter being derived from MSCV retrovirus, and a DNA scaffold attachment region (SAR).

In another embodiment, the invention provides a method of increasing expression of a transgene in a retrovirally transduced eukaryotic resting cell, the method comprising a) transducing a eukaryotic cell with a retroviral vector, the retroviral vector comprising (i) a transgene operatively linked a promoter, said promoter being derived from MSCV, and (ii) a scaffold attachment region (SAR); and b) expressing the transgene.

In a further embodiment, the invention further provides a method for therapeutically or prophylactically administering the retroviral vector of the invention to human in an amount sufficient to prevent, inhibit or stabilize an infectious, cancerous, or deleterious immune disease.

In a still further embodiment, the invention also provides retrovirus particles containing the retroviral vector of the present invention and a cell line producing a retrovirus containing the retroviral vector of the present invention.

Surprisingly, although the MSCV backbone is known to be prone to transcriptional silencing (Challita et al., 1994, 1995), the present invention provides substantially increased expression of transgene(s) in retroviral vectors containing the MSCV backbone or, at minimum, a MSCV promoter.

### BRIEF DESCRIPTION OF THE FIGURES

FIG. 1 shows the experimental design.

FIG. 2A represents real time PCR 72 hours following transduction of CD34⁺ cells with different retroviral vectors (3 MPB donors). MoMLV supernatant gave significantly higher transgene marking than the other 3 vectors.

FIG. 2B represents NGFR expression among cell subsets 72 hours post retroviral transduction (day 6 of culture). The data is the mean of the same MPB samples as in A ±standard error of the mean (SEM). ▪ Total cells, [symbol] CD34⁺ cells, [symbol] CD14+ cells.

FIG. 2C represents end point PCR assay for IRES-DHFR (45 cycles) on individual LTC-CFC colonies.

FIG. 3: [symbol] MoMLV, M□NLV-SAR, MS [symbol] V1, MSCV▪SAR.

FIG. 3A represents FACS analysis of NGFR transgene expression following 5 week cultures of transduced CD34⁺ cells on murine SyS1 stromal line. Asterisks indicate that addition of SAR to MSCV1 significantly increased the percentage of total and CD14⁺ cells expressing NGFR, and MSCV1 gave a significantly higher percentage NGFR expression among CD19⁺ B lymphoid cells than MoMLV. FIG. 3B represents mean fluorescence intensity of NGFR expression following 5 week cultures of transduced CD34+cells on the murine SyS1 stromal line. Asterisks indicate significantly higher MFI among total cells and the CD 14⁺ myeloid subset when SAR is added to MoMLV, and among all cell subsets when SAR is added to MSCV1.

FIG. 4 represents MPB CD34⁺ cells were transduced using TFK and RetroNectin™. Immediately after transduction (day 3), 2×10⁵ cells were injected into individual fetal human bone grafts in irradiated SCID-hu bone mice. After 8 weeks the contents of the grafts were analyzed for the transgene expression among donor cells.

FIG. 4A represents percentage of donor cells which expressed NGFR transgene.

FIG. 4B represents mean fluorescence intensity (MFI) of NGFR transgene expression, i.e. average level of transgene expression per cell.

FIG. 5 represents FACS analysis of NOD/SCID marrow (BM) and peripheral blood (PB) for expression of transgene by engrafted human cells 6 weeks following injection of CD34⁺ cells transduced with MSCV1 ±SAR. The proportion of human cells with high transgene expression increased 4.3 fold in marrow and 13 fold in PB when SAR was added to MSCV1.

### DETAILED DESCRIPTION OF THE INVENTION

In a first aspect of the invention, the retroviral vectors of the invention may derive from MSCV vectors, which as broadly defined herein, may have, at a minimum, a variant LTR from PCMV, or a variant LTR from another virus, in which the binding site for a suppressor of LTR transcription is deleted, and a functional binding site for the Sp1 transcription factor is created. MSCV vectors as described herein also, at a minimum, may have the 5′ untranslated region of the d1587rev virus, or some other virus, which alleviates transcriptional block of MoMLV LTR in murine embryonic stem cells.

MSCV vectors as described herein include MSCV derivatives thereof. In its broadest sense, a derivative of the MSCV vector, when used herein, means a vector having a nucleotide sequence corresponding to the nucleotide sequence of MSCV, wherein the corresponding vector has substantially the same structure and finction as the MSCV vector. The percentage of identity between the substantially similar MSCV vector and the MSCV vector desirably is at least 80%, more desirably at least 85%, preferably at least 90%, more preferably at least 95%, still more preferably at least 99%. Sequence comparisons may be carried out using any sequence alignment algorithm known to those skilled in the art, such as Smith-Waterman sequence alignment algorithm (see e.g. Waterman, M. S. Introduction to Computational Biology: Maps, sequences and genomes. Chapman & Hall. London: 1995. ISBN 0-412-99391-0, or at http:www-hto.usc. edu/software/seqaln/index.html.

The MSCV vectors as broadly defined herein may also include vectors such as the MESV vector (variant LTR from PCMV, d1587rev primer-binding site substituted), the MND vector (myeloproliferative sarcoma virus enhancer, negative control region deleted, d1587rev primer-binding site substituted), the SFFVp vector (spleen focus forming virus enhancer, d1587rev primer-binding site substituted) and the FMEV vector (spleen focusforming virus enhancer, d1587rev primer-binding site substituted).

A specific MSCV vector used in the present invention (hereinafter MSCV1) has a variant LTR from PCMV (PCC4 embryonal carcinoma cell-passaged myeloproliferative sarcoma virus) (Hawley et al., 1994, Pawliuk et al., 1997). One may add to MSCV1 the hIFN-β SAR to prolong transgene expression (Agarwal et al., 1998), which has already been demonstrated within the MoMLV backbone in primary T cells and macrophages in vitro (Auten et al., 1999). Human IFN-β SAR appears, within the present invention, to modulate the methylation of retroviral transgenes, and improve long-term expression at high levels in a copy number-dependent, position-independent manner.

In another aspect of the invention, the retroviral vector comprises a promoter derived from MSCV, which as broadly defined herein refers to expression control sequences which derives from MSCV vector, including MESV, MND, SFFVp and FMEV vectors. It means in particular that the promoter has a nucleotide sequence corresponding to the nucleotide sequence of any MSCV promoter, wherein the corresponding promoter has substantially the same structure and function as the MSCV promoter. Selection of expression control sequences is dependent on the vector selected, and may be readily accomplished by one of ordinary skill in the art. Examples of expression control sequences include a transcriptional promoter and enhancer, or RNA polymerase binding sequence, splice signals, polyadenylation signals including a translation initiation signal.

Additionally, depending on the host cell chosen and the vector employed, other genetic elements, such as additional DNA restriction sites, enhancers, sequences conferring inducibility of transcription, i.e. tissue or event specific, and selectable markers, may be incorporated into the retroviral vector. Preferably the retroviral vector of the present invention is replication defective.

In a further aspect of the invention, the retroviral vector of the present invention comprises DNA scaffold attachment region (SAR), i.e. “SAR elements”, which as broadly defined herein, refers to DNA sequences having an affinity or intrinsic binding ability for the nuclear scaffold or matrix. These elements are usually 100 to 300 or more base pairs long, and may require a redundancy of sequence information and contain multiple sites of protein-DNA interaction.

SAR elements are DNA elements which bind to the isolated nuclear scaffold or matrix with high affinity (Cockerill and Garrard, 1986, Gasser et al., 1986). Some of the SAR sequences have been shown to have enhancer activities (Phi-Van et al., 1990, McKnight et al., 1992), and some serve as cis-acting elements, driving B-cell specific demethylation in the immunoglobulin k locus (Lichtenstein et al., 1994, Kirillov, A. et al., 1996). The hIFN-β SAR element inhibits de novo methylation of the 5′ LTR, and appears to insulate the transgene from the influence of the flanking host chromatin at the site of retroviral integration. Position effects are thus decreased, resulting in sustained transgene expression in the T cell line CEMSS. Two to ten-fold enhancing effects on transgene expression by HIFN-β SAR addition to the MoMLV backbone have been described for primary T cells and macrophages (Agarwal et al., 1998, Auten et al. 1999).

Suitable SAR elements for use in the invention are those SAR elements which inhibit methylation of the 5′ LTR of the retroviral vector.

SAR elements may be obtained, for example, from eukaryotes, including mammals, plants, insects, and yeast. Mammals are preferred. Examples of suitable protocols for identifying SAR elements for use in the present invention are described in WO9619573 (Cangene Corp.), the disclosure of which is incorporated herein by reference.

In a preferred embodiment, more than one SAR element is inserted into the retroviral vector of the invention. Preferably, the SAR elements are located in flanking positions both upstream and downstream from the transgene and the operatively linked expression control sequence. The use of flanking SAR elements in the nucleic acid molecules may allow the SAR elements to form an independent loop or chromatin domain, which is insulated from the effects of neighbouring chromatin.

In another aspect of the present invention, the retroviral vector comprises any transgene of interest that is not found in the corresponding naturally occurring (i.e. wild-type) vector, which may be operably linked to the above listed MSCV promoters, for instance.

These nonnative genes can be desirably either a therapeutic gene or a reporter gene, which, preferably, is capable of being expressed in a cell entered by the retroviral particle. A therapeutic gene can be one that exerts its effect at the level of RNA or protein. For instance, a protein encoded by a therapeutic gene can be employed in the treatment of an inherited disease, e.g., the use of a cDNA encoding the cystic fibrosis transmembrane conductance regulator in the treatment of cystic fibrosis. Further, the protein encoded by the therapeutic gene can exert its therapeutic effect by causing cell death. For instance, expression of the protein, itself, can lead to cell death, as with expression of diphtheria toxin A, or the expression of the protein can render cells selectively sensitive to certain drugs, e.g., expression of the Herpes simplex thymidine kinase gene renders cells sensitive to antiviral compounds, such as acyclovir, gancyclovir and FIAU (1-(2-deoxy-2-fluoro-.beta.-D-arabinofuranosil)-5-iodouracil). Alternatively, the therapeutic gene can exert its effect at the level of RNA, for instance, by encoding an antisense message or ribozyme, a protein that affects splicing or 3′ processing (e.g. polyadenylation), or a protein that affects the level of expression of another gene within the cell, e.g. by mediating an altered rate of mRNA accumulation, an alteration of mRNA transport, and/or a change in post-transcriptional regulation. Thus, the use of the term “therapeutic gene” is intended to encompass these and any other embodiments of that which is more commonly referred to as gene therapy as known to those of skill in the art. The term “therapeutic agent” is used in a generic sense and includes treating agents, prophylactic agents, and replacement agents.

One clinical trial used a MoMLV vector containing RevM10 anti-HIV transgene (Malim et al., 1989). There appeared to be a threshold level for the RevM10 protein to allow efficient competition with the normal HIV Rev protein (Plavec et al., 1992). In one particular embodiment of the present invention, MSCCV-SAR vector thus expresses RevMl 0 and/or an antisense of the HIV reverse polyrnerase in order to obtain an increased level of in vivo RevM10 and/or antisense production per cell.

In a further aspect of the present invention, there is provided a method for increasing expression of a transgene in a retrovirally transduced eukaryotic resting cell, the method comprising a) transducing a eukaryotic cell with a retroviral vector, the retroviral vector comprising (i) a transgene operatively linked a promoter, said promoter being derived from MSCV, and (ii) a scaffold attachment region (SAR); and b) expressing the transgene.

In a further aspect of the present invention, the invention provides a method for therapeutically or prophylactically administering a retroviral vector of the invention to human in need thereof in an amount sufficient to prevent, inhibit, or stabilize an infectious, cancerous, neuronal, or deleterious immune disease. Viral and cancer diseases are preferred diseases as proofs of concept have been well established.

In a further aspect of the present invention, a cell line is provided which produces a retrovirus of the present invention. Illustrations of cell lines that can be developed for this purpose are found in the following listing of references.

The following example 1 demonstrates the effect of HIFN-β SAR within two different retroviral backbones, in long term assays for HSC. The following was utilized: a) 5 week stromal cultures (Murray et al., 1999b), b) human HSC repopulation of SCID-hu bone grafts at 8 weeks (Murray et al., 1995, Luens et al., 1998) and c) human HSC repopulation of NOD/SCID mice at 6 weeks (Wang et al., 1997). The predominant effect of addition of SAR to MoMLV or MSCV1 backbones was to increase the mean fluorescence intensity (MFI) of transgene expression. Among the four vectors tested, MSCV1-SAR gave the highest percentage of transgene expressing cells in stromal cultures and SCID-hu bone assays. Use of MSCV1-SAR vectors may optimize the level of therapeutic transgene expression among HSC progeny in vivo.

The invention now will be described with respect to the following examples. It is to be understood that the scope of the present invention is not to be limited to the specific embodiments described above. The invention may be practiced other than particularly described and still be within the scope of the accompanying claims.

The following references are incorporated herein in their entirety:

U.S. Ser. No. 09/194,301 entitled “Vectors comprising SAR element” to Agarwal, et al. filed Nov. 23, 1998.

U.S. Pat. No. 5,707,865 entitled “Retroviral vectors for expression in embryonic cells”

AGARWAL, M., AUSTIN, T. W., MOREL, F., CHEN, J., BOHNLEIN, E., PLAVEC, I. (1998). J. of Virol. 72: 3720-3728

AUTEN, J., AGARWAL, M., CHEN, J., SUTTON, R., PLAVEC, I. (1999). Human Gene Therapy 10:1389-1399

BURNS, J. C., T. FRIEDMANN, W. DRIEVER, M. BURRASCANO, and YEE, J. K. (1993). Proc. Natl. Acad. Sci. USA. 90: 8033-8037.

CHALLITA, P. M., KOHN, D. B. (1994) o. Proc. Natl. Acad. Sci. USA 91: 2567-71.

CHALLITA, P-M., SKELTON, D., EL-KHOUEIRY, A., YU, X. -J., WEINBERG, K., KOHN, D. B. (1995). J. of Virology 69: 748-755.

CHENG, L., DU, C., LAVAU, C., CHEN. S., TONG, J., CHEN, B. P., SCOLLAY, R., HAWLEY, R. G., HILL, B. (1998). Blood 92: 1-11.

COCKERILL, P. N. and GARRARD, W. T. (1986). Cell 44: 273-282.

CWIRLA, S. E et al. (1997). Science 276: 1696-1699

FLANAGAN, J. R., KRIEG, A. M., MAX, E. E., KHAN, A. S. (1989). Mol. Cell Biol. 9: 739-746

FORESTELL, S. P. et al.(1995). Gene Ther. 2: 723-730

FORESTELL, S. P., DANDO, J. S., CHEN, J., de VRIES, P., BOHNLEIN, E., RIGG, R. J. (1997). Gene Therapy 4: 600-610

GASSER, S. M. and LAEMMLI, U.K. (1986). Cell 46: 521-530

GERARD, C. J., OLSSON, K., RAMANATHAN, R., READING, C., HANANIA, E. G. (1998). Cancer Research 58: 3957-3964

HANENBERG, H., HASHINO, K, KONISHI, H., HOCK, R. A., KATO, I, WILLIAMS, D. A. (1997). Human Gene Therapy 8: 2193-2206

HAWLEY, R. G., FONG, A. Z. C., BURNS, B. F., HAWLEY, T. S. (1992). J Exp Med 176: 1149-1163

HAWLEY, R. G., LIEU, F. H. L., FONG, A. Z. C., HAWLEY, T. S. (1994). Gene Therapy 1: 136-138.

JANG, S. K. et al. (1989). J. Virol. 63:1651-1660.

KIRILLOV, A., KISTLER, B., MOSTOSLAVSKY, R., CEDAR, H., WIRTH, T., BERGMAN, Y. (1996). Nat. Genet. 13: 435-441

KYOIZUMI, S, BAUM, C. M., KANESHIMA, H., McCUNE, J. M., YEE, E. J., NAMIKAWA, R. (1992). Blood 79:1704

LICHTENSTEIN, M., KEINI, G., CEDAR, H., BERGMAN, Y. (1994). Cell 76: 913-923

LU, M., ZHANG, N., MARUYAMA, M., HAWLEY, R. G., HO, A. D. (1996). Human Gene Therapy 7: 2263-2271

LUENS, K. M., TRAVIS, M. A., CHEN, B. P., HILL, B. L., SCOLLAY, R., MURRAY, L. J. (1998). Blood 91:1206-1215

MALIM, M. H., BOHNLEIN, S., HAUBER, J., CULLEN, B. R. (1989). Cell 58:205-214

McKNIGHT, R. A., SHAMAY, A., SANKARAN, L, WALL, R. J., HENNIGHAUSEN, L (1992). Proc. Natl. Acad. Sci. USA 89: 6943-6947

MILLER, A. D., and G. J. ROSMAN (1989). BioTechniques. 7: 980-990.

MORITZ, T., PATEL, V. P., WILLLAMS, D. A. (1994). J Clin Invest 93:1451-1457

MURRAY L. et al. (1995). Blood 85: 368-378

MURRAY L. J., YOUNG J. C., OSBORNE, L. J., LUENS K. M., SCOLLAY, R., HILL, B. L. (1999a). Exp. Hematol. 27: 1019-1028

MURRAY, L., LUENS, K., TUSHINSKI, R, JIN, L., BURTON, M., CHEN, J., FORESTELL, S, HILL, B. (1999b). Human Gene Therapy 10: 1743-1752

OLSSON, K., GERARD, C. J., ZEHNDER, J., JONES, C., RAMANATHAN, R., READING, C., HANANIA, E.G. Novel real-time t(11;14) and t(14;18) PCR assays provide sensitive and quantitative assessments of minimal residual disease (MRD) Leukemia (In press)

PETERSEN, R., KEMPLER, G., BARKLIS, E. (1991). Mol. Cell Biol. 11: 1214-1227

PHI-VAN, L., von KRIES, J. P., OSTERTAG, W., STRATLING, W. H. (1990). Mol. Cell Biol. 10: 2302-2307

PLAVEC, I., AGARWAL, M., HO, K. E., PINEDA, M., AUTEN, J., BAKER, J., MATSUZAKI, H., ESCAICH, S., BONYHADI, M., BOHNLEIN, E (1997). Gene Therapy 4: 128-139

RIGG, J. R., CHEN, J., DANDO, J. S., FORESTELL, S. P., PLAVEC, I., BOHNLEIN, E. (1996). Virology 218: 290-295

SIMONSEN, C. C., and LEVINSON, A. D. (1983). Proc. Natl. Acad. Sci. USA. 80: 2495-2499.

WANG, J. C. Y., DOEDENS, M., DICK, J. E. (1997). Blood 89: 3919-3924

YOUNG, J. C., LIN, K., HANSTEEN, G., TRAVIS, M., MURRAY, L. J., JAING, L., SCOLLAY, R., HILL, B. L. (1999). Exp. Hematol. 27: 994-1003

### EXAMPLE 1

The goal of the present example was to investigate whether retroviral vector modification could increase the level of transgene expression in vivo among the progeny of engrafted HSC derived from human MPB. An alternative to the MoMLV backbone, known to be prone to transcriptional silencing (Challita et al., 1994, 1995), is the MSCV1 backbone (Hawley et al. 1992, 1994). In vitro studies have indicated that the MSCV1 LTR may be more active than the MoMLV LTR in hematopoietic cells (Lu et al., 1996, Cheng et al., 1998). We wished to compare these two retroviral backbones, with or without addition of the HIFN-β SAR element, for transduction of human mobilized CD34⁺ cells, using in vivo HSC repopulation models.

Materials and Method

Construction of Retroviral Vectors

The retroviral vectors MoMLV (LNiD) and MoMLV1-SAR (LNiDS) have been described previously (Auten et al., 1999). The vectors are MoMLV-based (Miller and Rosman, 1989) and contain the murine dihydrofolate reductase (DHFR) selectable marker gene (Simonsen and Levinson, 1983), and the truncated nerve growth factor receptor (NGFR) gene. Expression of the DHFR gene is mediated by the internal ribosomal entry site (IRES) from the encephalomyocarditis virus (Jang et al., 1989). The LNiDS vector has the interferon-β SAR sequence (Agarwal et al., 98) inserted just upstream of the 3′ LTR (LTR-NGFR-IRES-DHFR-[±SAR] -LTR). Vectors MSCV1 (MSCVNiD) and MSCV1-SAR (MSCVNiDS) have a structure equivalent to the LNiD and the LNiDS vectors, only the vector backbone sequence was derived from MSCV1 (Hawley et al., 1992, 1994). Retroviral vector plasmid DNAs were co-transfected with a VSV-G expression plasmid (Burns et al., 1993) into gp47 cells as described (Rigg et al., 1996). Forty-eight hours post-transfection, culture supernatants were used to inoculate amphotropic ProPak-A packaging cells (Rigg et al., 1996). Following transduction, transgene-expressing ProPak-A cells were enriched by selection in 200 nM trimetrexate (US Bioscience, West Conshohocken, Pa.) to generate polyclonal producer cell populations. Amphotropic retroviral vector supernatants were produced from human ProPak-A.6 cells in serum-containing medium in a packed-bed bioreactor in perfusion mode, as previously described (Forestell, 1997), and kept frozen at −80° C. All producer cells tested negative for replication competent retrovirus by S⁺L assay on PG-13 cells (Forestell et al., 1995).

Cells

Leukapheresis samples were obtained from normal donors mobilized with 7.5 or 10 μg/kg/day of G-CSF for 4-5 days at the Department of Medicine, Roswell Park Cancer Institute, Buffalo, N.Y. (Dr. P. McCarthy), Stanford Hospital (Dr. R. Negrin) or the AIDS Community Research Consortium, Redwood City, Calif. (Dr. B. Camp). Donors signed informed consent forms according to local IRB requirements. CD34⁺ cells were enriched at SyStemix using Isolex 300SA (Baxter Healthcare Corp., Deerfield, Illinois).

Cytokines

Cytokines used for retroviral transduction included TPO mimetic peptide AF13948 (50 ng/ml) (based on the sequence published by Cwirla et al., 1997 and synthesized by SynPep, Dublin, Calif.), flt3 ligand (100 ng/ml) and kit ligand (100 ng/ml) (SyStemix, Palo Alto, Calif.). This cytokine combination will be referred to as TFK. Other cytokines used for assays included interleukin-3 (IL-3), IL-6 (10 ng/ml) and leukemia inhibitory factor (LIF) (Novartis Inc., Basel, Switzerland) at 100 ng/ml, GM-CSF at 10 ng/ml, and erythropoietin at 2U/ml (both clinical grade).

Retroviral Infection of MPB CD34⁺ Cells by Culture on RN

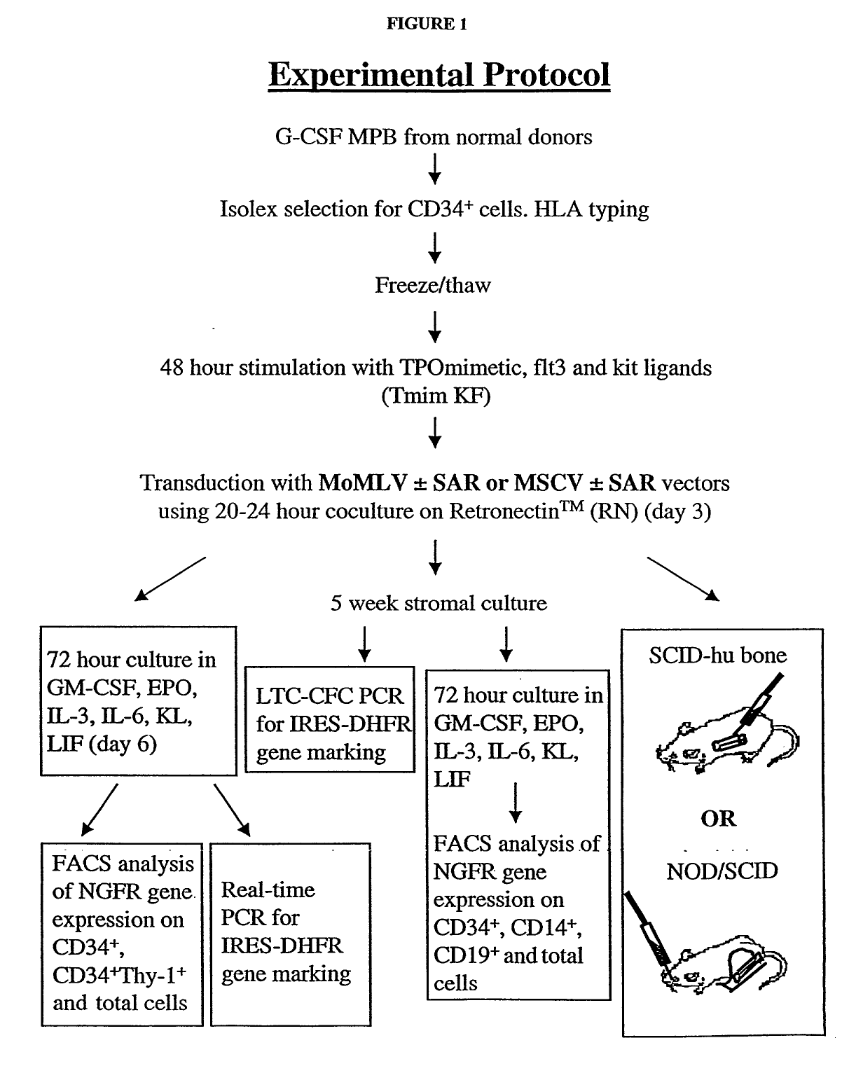

The experimental protocol is summarized in FIG. 1. MPB were cultured at 10⁶ cells per ml (5×10⁶ for each vector) in X Vivo 15 medium (BioWhittaker, Walkersville, Md.) containing the TFK cytokine combination for 48 hours (hr) at 37° C., 5% CO₂. They were then incubated with retroviral supernatant on RetroNectin™ (BioWhittaker) coated plates (non tissue culture-treated, Falcon) for 20-24 hour culture at 37° C. in 5% CO₂, as previously described (Murray et al., 1999b). Cells were then removed from the plates by vigorous pipetting and centrifuged. Cell pellets were resuspended in X Vivo 15, and viable cells were counted by trypan blue exclusion.

Development of a Quantitative PCR Assay for IRES-DHFR Junction

A real-time PCR assay targeting the IRES-DHFR junction was developed as previously described for t(14;18) and t(11;14) sequences (Olsson et al., In press).

Quantitation of Transgene Delivery to CD34⁺ cells 72 Hours Following Transduction

The IRES-DHFR and 13-actin real-time PCR assays were used to quantitate and compare the percentage of gene delivery with different vectors. Cells from three different MPB samples were frozen 72 hours following transduction, and genomic DNA was later extracted and quantitated as previously described (Olsson et al. In press). Both quantitative PCR assays amplified less than or equal to 0.3 μg (50,000 cell equivalents) of purified DNA in each 50 μl reaction. Reaction components for the IRES-DHFR PCR included 1× TaqMan Buffer, 3.5 mM MgCl₂, 0.2 mM each of dATP, dCTP and dGTP, 0.4 mM dUTP, 0.65 μM forward primer:

(5′-CGATGATAAGCTTGCCACAACCAT-3′), 0.5 μM reverse primer:

(5′-AGCGGAGGCCAGGGTAGGTCT-3′), 0.2 μM probe:

(5′-TTCGACCATTGAACTGCATCGTCGCC-3′), 1.5 U TaqGold, 0.5 U uracil N-glycosylase (UNG) and 5% dimethyl sulfoxide (Sigma Chemical Co., St. Louis, Mo.) in sterile water (Baxter Healthcare). The β-actin reaction mix was prepared according to the previously published protocol (Gerard et al., 1998). Oligonucleotide primers and probes were synthesized by the Oligo Factory (Perkin-Elmer) and PCR reagents were obtained from Perkin-Elmer Corporation (Norwalk, Conn.). Both IRES-DHFR and 13-actin PCR assays were performed on the same plate in an ABI PRISM 7700 Thermal Cycler (Perkin-Elmer) and data acquired in the DHFR-IRES PCR were normalized to the quantities estimated by β-actin PCR. Cycling conditions included a 2 min 50° C. incubation, a 10 min 95° C. incubation, and 45 cycles of a 15 second (sec) 95° C. denaturation and a 1 min 62° C. annealing step. Reactions containing experimental samples, standards, 10 mM Tris (pH 8.0) or no-template controls were run concurrently. The standards for both assays were prepared using DNA derived from an MSCV-SAR transduced CEMSS T cell-line, trimetrexate-selected to 99.3% NGFR expression (CEMSS+) diluted into 10 mM Tris (pH 8.0). The initial standard (containing 100% CEMSS+ DNA at a 0.03 μg/μl concentration) was serially diluted 1:5 to prepare Standards B through D, and Standards E through G were prepared by serially diluting Standard D 1:10. Diluting into no-template DNA did not significantly change PCR efficiency and reduced the linear range of β-actin PCR (data not shown).

Determination of Percent Transgene Delivery to LTC-CFC by Endpoint PCR

Cells were harvested from 5 week stromal cultures. Triplicate aliquots of 40,000 cells were placed into 1 ml methylcellulose colony assays (MethoCult, StemCell Technologies, Vancouver, Canada) with GM-CSF, EPO, IL-3, IL-6 and kit ligand. Sixty four long term culture derived colony-forming cells (LTC-CFC) colonies were individually picked by pipette and dispensed into 50 μl of PCR lysis buffer (Murray et al., 1999). Plates were incubated overnight at 37° C. and heat inactivated at 95° C. for 15 minutes, before storage at −80° C. Ten μl of each lysate were placed in 40 μl of the IRES-DHFR reaction mix described above. PCR was performed in the ABI PRISM 7700 Thermal Cycler (Perkin-Elmer) using the quantitative PCR cycling protocol with a shortened 30 sec 62° C. annealing step. Presence of the IRES-DHFR transgene sequence was assessed by scoring the percentage of samples with detectable fluorescence increase. In order to prepare the standards used in the end-point PCR, CEMSS wild type (CEMSS−) and CEMSS+ cells were washed in PBS, centrifuged at 1300 rpm for 5 minutes, and resuspended in PBS to a concentration of 1000 cells perμl. Cells were then aliquotted into six 1.5 ml tubes such that each tube contained 2.5×1 total cells and decreasing numbers of transduced (CEMSS+) cells. Standards A through E contained 100 to 1% CEMSS+) cells, and Standard F served as a CEMSS negative control.

NGFR Transgene Expression among Progeny of PHP from 5 Week Stromal Cultures

Twenty four hours after initial exposure to retrovirus, cells were counted (day 3). For each retroviral vector test sample, duplicate cultures per condition were plated on top of SyS1 murine stromal cells (twenty thousand cells per well) in 24-well plates (Corning Science Products, Acton, Mass.) for 5 week culture in the presence of exogenous human IL-6 and LIF. Following these long term stromal cultures, the expression of NGFR transgene among cell subpopulations was analyzed as previously described (Murray et al., 1999b).

SCID-hu Bone Repopulation Assay

The SCID-hu bone assay (Kyoizumi et al., 1992, Murray et al., 1995) was performed by irradiating SCID-hu bone mice with 350 rads, and injecting 2×10⁵ cells (post-transduction CD34⁺ cells on day 3) directly into individual fetal human bone grafts, which were HLA-mismatched with the CD34⁺ donor cells. After 8 weeks, mice were sacrificed and the cells in the human bone piece analyzed for human cells (W6/32 positive), donor cells (HLA marker positive) and NGFR transgene expression. In 4 preliminary experiments, MoMLV-SAR and MSCV1-SAR vectors were compared. In 5 further experiments, all 4 vectors were compared simultaneously to determine the role of the SAR element within each vector backbone. In total, NGFR transgene expression of donor cells was analyzed for the following number of different MPB samples: eight for MoMLV-SAR, nine for MSCV1-SAR, four for MoMLV, three for MSCV1.

NOD/SCID Mouse Repopulation Assay

Six to ten week old NOD/SCID mice (Jackson Labs derived, and bred at SyStemix) were irradiated with 350 rads, before injection into the orbital sinus of 10-20 million CD34⁺ cells (in 100 μl), immediately following transduction. Six weeks later, the mice were sacrificed, and peripheral blood cells, plus marrow cells from the long bones of the hind limbs were recovered. Cell suspensions were lysed to remove red blood cells and analyzed for transgene expressing human cells, by staining with combinations of three of the following antibodies: anti-CD45-APC, anti-CD34-FITC, anti-CD19-FITC, anti-CD33-FITC, anti-CD14-FITC (Becton Dickinson), and anti-NGFR-PE. Cells were analyzed on a FACS Calibur™.

Statistical analysis

The Mann Whitney t test (non-paired, 2 tailed) was used to calculate the significance of the differences between two vectors using a PRIZM program. Differences were considered statistically significant when P<0.05.

Results

Real-time PCR Quantitation of Transgene Delivery to CD34⁺ Cells 72 Hours Following Retroviral Transduction

Comparison of retroviral vector supernatants by end-point titer is a poor predictor of the efficiency of gene transduction of primary cells (Forestell et al., 1995). A quantitative PCR assay was thus developed to measure the level of transgene delivery to the total cell population 72 hours following retroviral transduction. The assay was based on a sequence sparming the IRES-DHFR junction, common to all four vectors. A logarithmic increase in fluorescence (ΔRₙ) was observed for serially diluted CEMSS cells transduced with MSCV1-SAR vector in a 50,000 cell background (detection limit of 4 cell equivalents, 0.008%). No logarithmic increase could be observed when untransduced CD34⁺ cells were used (data not shown). Standard curves were generated from regression analysis of the cycle number at which samples' ΔRₙ values exceeded a user-defined threshold versus starting copy number, and could be used to estimate DNA concentrations in test samples (day 6 cultures of transduced CD34+cells). Correlation coefficients were always >0.99. IRES-DHFR PCR data was normalized to the quantities of DNA estimated by PCR for β-actin.

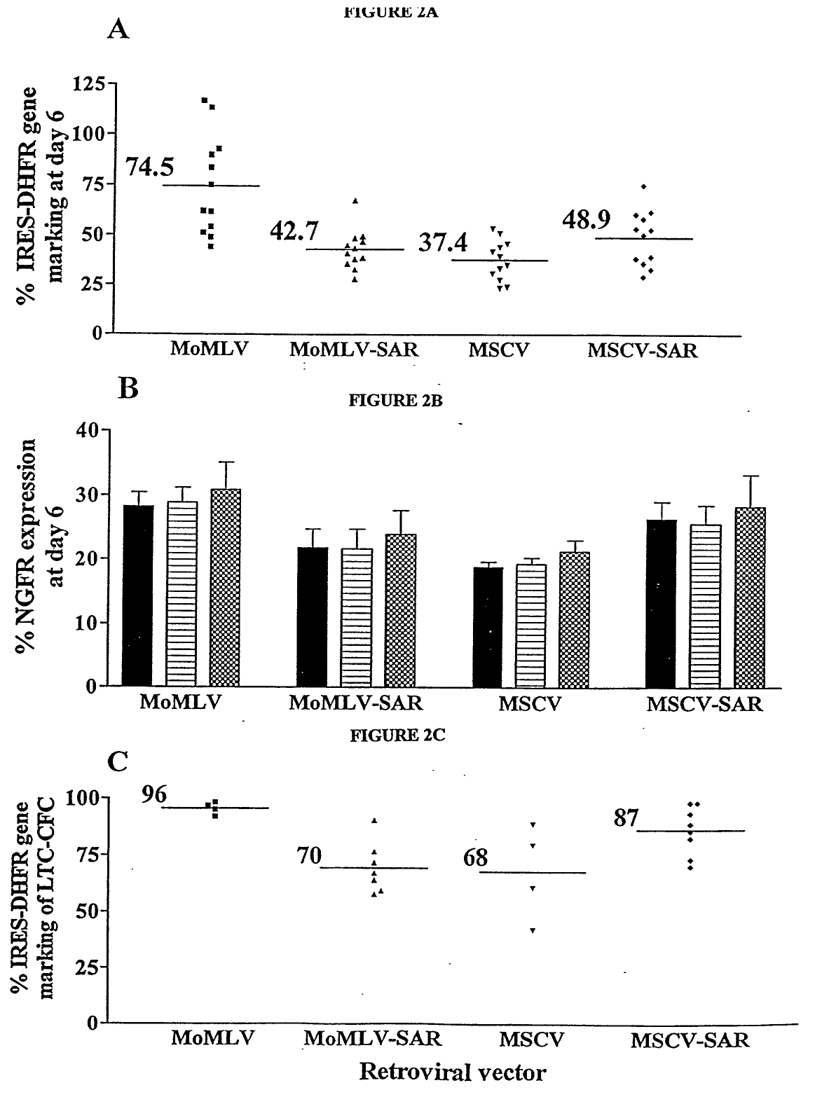

DNA was extracted from CD34⁺ cells from three different MPB donors 72 hours post-transduction. The samples analyzed were a subset of the donors assayed in the SCID-hu bone model. Samples were run four times in duplicate to determine the mean percentage of cells marked with IRES-DHFR transgene at the beginning of the long term assays (FIG. 2A). Transgene marking was not significantly different for MoMLV-SAR, MSCV1 and MSCV1-SAR vectors (37-49%). However, the MoMLV supernatant appeared to be of superior quality, since it gave significantly higher mean transgene marking of 74.5%.

NGFR Expression 72 Hours Following Retroviral Transduction

FIG. 2B shows the comparison of NGFR expression at day 6 for the same three MPB CD34⁺ samples tested by PCR. The ratio of NGFR expression among total cells over IRES-DHFR gene marking was not significantly different for MSCV1 and MSCV1-SAR (0.48 and 0.52, respectively). For MoMLV, the ratio was only 0.35, compared to 0.45 for MoMLV-SAR, i.e. the proportion of marked cells with transgene expression was lower for MoMLV at this early timepoint.

End-point PCR Analysis of DHFR Gene Marking of LTC-CFC

Comparison of the percent gene delivery to primitive LTC-CFC is shown in FIG. 2C. MoMLV (96%)>MSCV1-SAR (87%)>MoMLV-SAR (70%)>MSCV1 (68%). The maximum difference in gene marking of primitive hematopoietic progenitors from our original CD34⁺ cell populations, was thus 1.4 fold.

NGFR Transgene Expression Among Progeny of Primitive Hematopoietic Progenitors Following 5 Week Stromal Culture

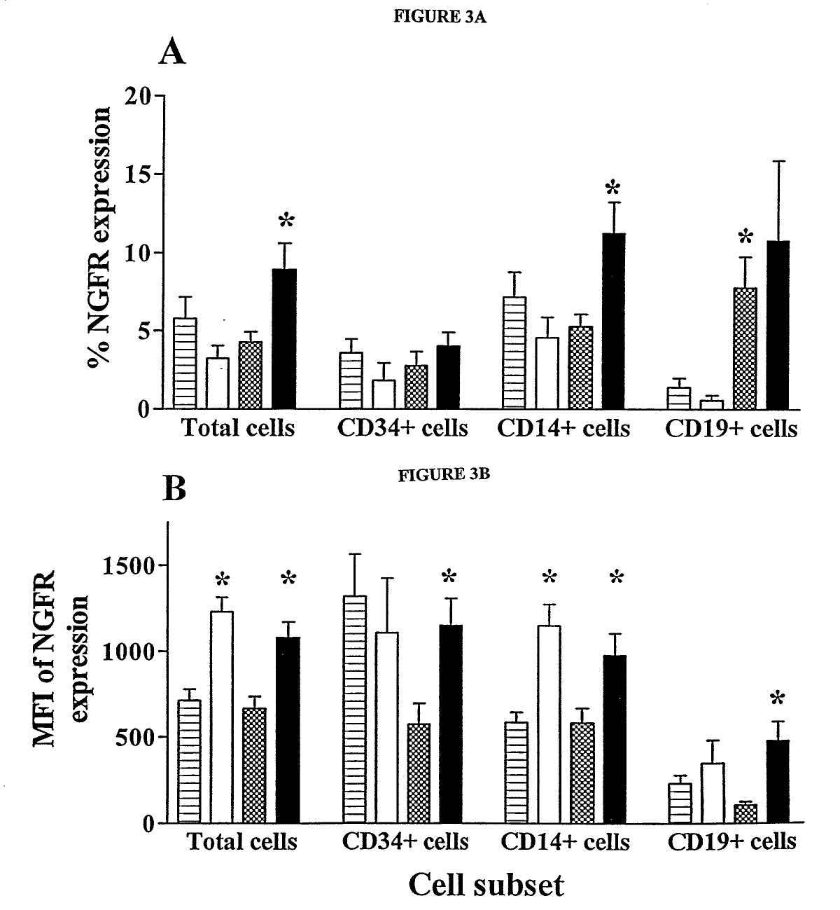

Analysis of NGFR expression of cell subsets harvested from 5 week stromal cultures is shown in FIG. 3A (n=6). Comparing the vector backbones in the absence of SAR, the only significant difference was NGFR expression by a much higher percentage of B lymphoid cells using MSCV1 (7.8%) versus MoMLV vector (1.4%) (P=0.009). Addition of SAR to MoMLV did not increase the percentage of total cells expressing NGFR (3.2% v 5.8% of total cells). The lower percentage with MoMLV-SAR could have been due to lower transgene marking (FIG. 2A). However, addition of SAR to MSCV1 vector increased the percentage of total cells expressing NGFR from 4.3 to 9%, predominantly an effect on the CD14⁺ myelomonocytic cells (P=0.015).

Mean Fluorescence Intensity (MFI) of NGFR Expression Following 5 Week Stromal Culture

Addition of SAR significantly increased the MFI of transgene expression in both vector backbones: 1.7 fold for MoMLV and 1.6 fold for MSCV1 (FIG. 3B). For MoMLV, a significant 2 fold increase of MFI was observed for the CD14⁺ myelomonocytic population (P=0.0012). For MSCV1, the SAR effect was significant for all cell subsets: 2 fold for CD34⁺, 1.7 fold for CD14⁺ and 4.6 fold for CD19⁺ cells (P<0.009).

Transgene Expression among Progeny of Engrafted HSC in SCID-hu Bone

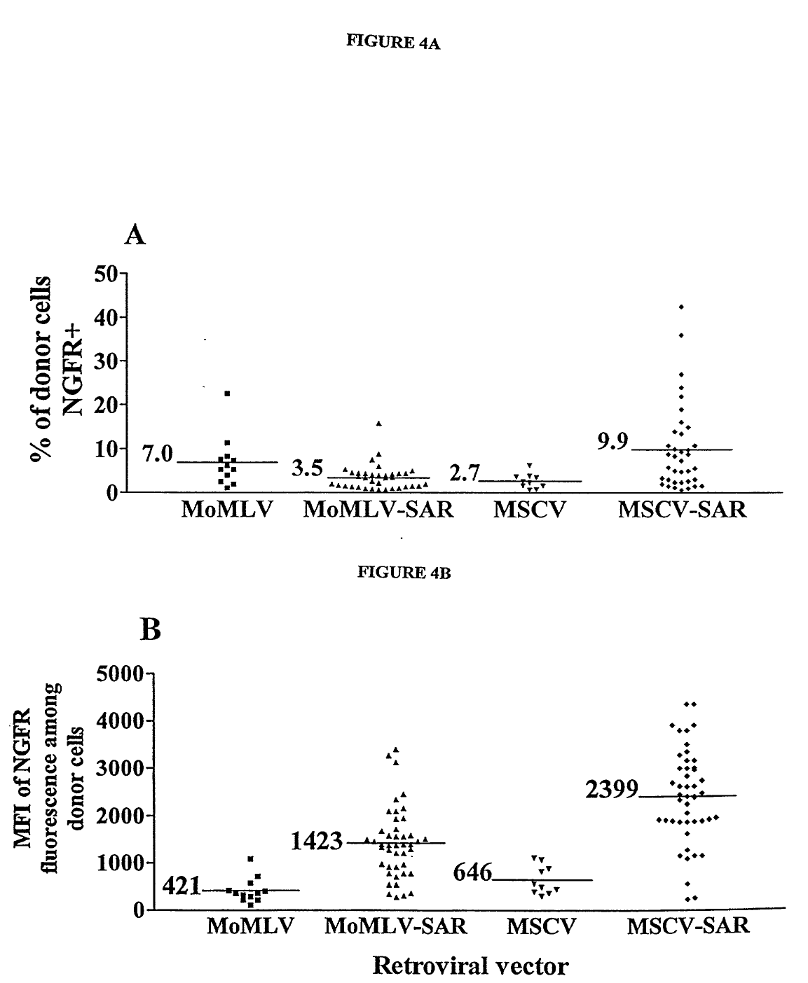

Addition of SAR to MoMLV backbone did not increase the percentage of donor cells expressing transgene in SCID-hu bone grafts (FIG. 4A). The apparently two fold higher percentage of NGFR expression among progeny of MoMLV transduced cells (7%) compared to MoMLV-SAR transduced cells (3.5%) could be due to the higher level of transgene marking at day 6 with the MoMLV supernatant (FIG. 2A). Indeed, if we normalize for transgene marking post transduction to make the other 3 vectors equivalent to MSCV1-SAR, the expression from MoMLV transduced cell progeny is almost the same as MoMLV-SAR (4.6% v 3.9%).

Addition of SAR to MSCV1 backbone increased the percentage of donor cells expressing transgene from 2.7 to 9.9% (P<0.0001) as shown in FIG. 4A. Insufficient cells could be recovered from bone grafts to perform PCR assays to determine whether this higher percent expression was due to higher levels of transgene marking with MSCV1-SAR. The predominant effect of SAR was to increase the MFI of NGFR expression among donor cells 3.4-3.7 fold (FIG. 4B).

Transgene Expression among Progeny of Engrafted HSC in NOD/SCID Mice

NOD/SCID Repopulation Assays were Performed to Compare:

A. MoMLV-SAR with MSCV1-SAR

Engraftment of cultured human CD34+cells in the NODISCID mouse marrow has shown a high degree of variability among different MPB samples—from 0 to 90% human cells for 10 million cells injected. Our first experience in the current series of experiments was that 5 million transduced cells gave only 1-4% human cell engraftment in the marrow of ⅓ mice. For MoMLV-SAR, 8.7% of human cells, and for MSCV1-SAR, 14% of human cells expressed NGFR in mouse marrow (data not shown).

In a second experiment using 10 or 20 million transduced cells, more consistent engraftment was achieved, and results are summarized in Table 1. Twelve out of twelve mice engrafted with 41-82% human cells in the bone marrow, and 10/12 mice had human cells in peripheral blood (PB) at 15-52%. Using MSCV1-SAR vector, we observed a mean of 4.3% NGFR expression among human cells in marrow (15.4 fold higher than with MoMLV-SAR, P=0.002). In PB, a mean of 5% of human cells expressed NGFR (4 fold higher than with MoMLV-SAR, P=0.038).

B. MSCVI-SAR with MSCV1

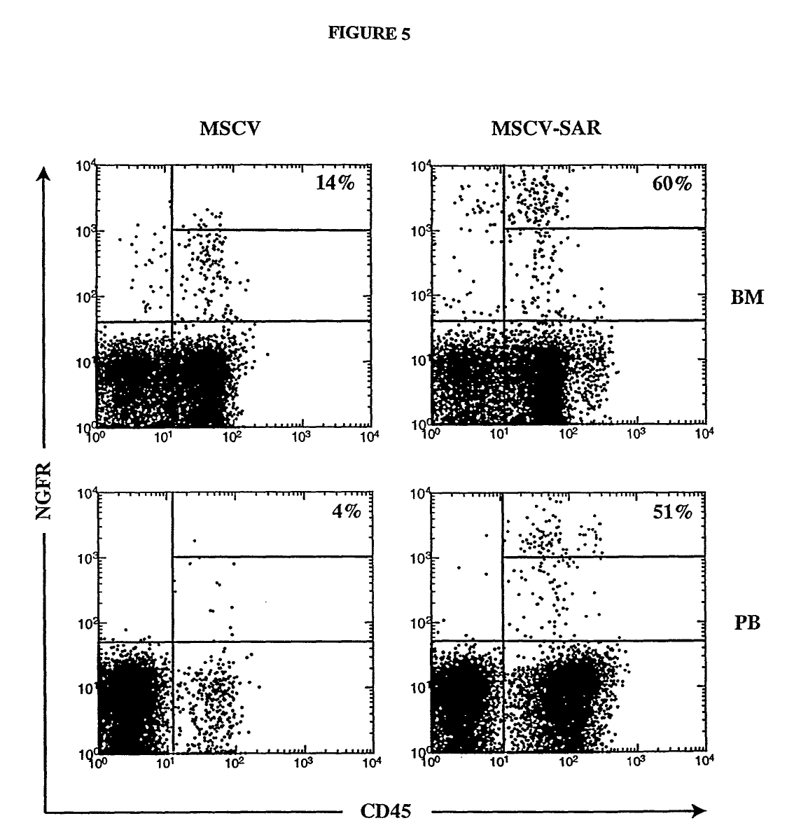

Ten million CD34⁺ cells from one MPB donor were injected post transduction with either MSCV1 or MSCV1-SAR vectors into each NOD/SCID mouse, to determine the role of the SAR element. All eight mice engrafted human cells in the mouse marrow (about 40%), and six out of eight engrafted human cells in the PB (about 15%) (Table 2A). Addition of SAR to MSCV1 gave a 2.2 fold higher percentage of human cells expressing transgene (3.63% versus 1.63%). With the SAR element present, expression was 3.2% in the PB, compared to only 1.2% with MSCV1. In this experiment we harvested sufficient cells from the mouse marrow to quantitate the percentage of human cells bearing the IRES-DHFR sequence among the progeny of repopulating HSC. 100% of MSCV1 and 81% of MSCV-SAR transduced cells, which were IRES-DHFR marked, also expressed NGFR. The range of gene marking of human cells was 0.7-2.2% for MSCV1 and 2.7-5.8% for MSCV-SAR. The increased percentage of human cells expressing NGFR when SAR is added to MSCV1 (2.2 fold) could, therefore, be explained by higher gene marking with MSCV1-SAR (2.7 fold) in this experiment. The predominant effect of addition of SAR to MSCV1 in the NOD/SCID repopulation model was to increase the MFI 2.9 fold for CD19⁺ B lymphoid cells, and 2.5 fold for CD33⁺ myeloid cells (Table 2B). The percentage of human cells with high level transgene expression (>10³ MFI) thus increased up to 61% of NGFR⁺ B cells (3 fold), and up to 29% of NGFR⁺ myeloid cells (7.4 fold). A representative FACS analysis, showing this high level transgene expression is shown in FIG. 5.

Discussion

We have analyzed the effect of hIFN-β SAR within both MoMLV and MSCV1 backbones in long term functional assays, which attempt to analyze the transgene expression in the progeny of human HSC in vivo. Since the quality of vector supernatants can vary, a sensitive, quantitative real-time PCR assay was developed to compare levels of IRES-DHFR transgene marking 72 hours following transduction. The SAR element appeared to have different effects in the two vector backbones with regard to the percentage of cells expressing transgene. Only when added to the MSCV1 backbone did SAR increase the percentage of NGFR positive cells: 2.1 -fold in vitro, and 2.2-2.8 fold in vivo. Using an optimized transduction protocol (TPO, flt3 and kit ligands and RetroNectin™) (Murray et al., 1999b) and the MSCV-SAR vector, about 11% of B lymphoid and CD14⁺ myelomonocytic cells, and 4% of CD34⁺ cells expressed NGFR post stromal culture. The high expression among B lymphoid cells appeared to be mostly a feature of the MSCV1 backbone itself (7.8% of B cells), while less than 1.4% of B cells expressed transgene using MoMLV ±SAR. Since LTC-CFC transgene marking did not differ more than 1.4 fold, it is likely that the high expression among B cells is due to modifications in the MSCV1 backbone, and is consistent with the study published by Cheng et al. (1998). However, MSCV1 did not give rise to a higher percentage transgene expression than MoMLV in the SCID-hu bone assay, which predominantly analyzes human HSC B lymphoid progeny. The percentage of CD14⁺ myeloid cells expressing NGFR was significantly increased by addition of SAR to MSCV1 (P=0.015). Using MSCV1-SAR, we can now observe a mean of about 10% of donor cells expressing transgene in the SCID-hu bone grafts. Real-time PCR could not be performed on cells from SCID-hu bone grafts to compare levels of transgene marking, due to recovery of insufficient cell numbers. The high levels of transgene expression seen in some grafts, using different MPB donors suggests that in spite of a high degree of variation, the probability of high percentage NGFR expression among donor cells is increased using MSCV1-SAR.

Perhaps most relevant to human gene therapy trials is the comparison of the percentage of human cells, which had detectable NGFR expression in the peripheral blood of NOD/SCID mice. MSCV1-SAR gave approximately 3-4 fold higher percentage of human PB cells expressing NGFR (3-5%), when compared to either MoMLV-SAR or to MSCV1. This higher percentage of NGFR expressing cells in vivo may be explained by higher transgene delivery to repopulating HSC (Table 2A). Further experiments confirmed that there is consistently a larger difference in transgene marking among HSC progeny between MSCV1-SAR and MSCV1 in vivo (2.7 fold) than among in vitro LTC-CFC (1.3 fold).

The predominant effect of SAR within both retroviral backbones was the increased level of transgene expressed per cell, as measured by the mean fluorescence intensity of NGFR expression. Addition of SAR to MoMLV resulted in a 2 fold greater MFI among CD14⁺ cells post stromal culture, in agreement with the study of Auten et al. (1999). Addition of SAR to MSCV1 had a more multilineage effect, increasing the MFI about 2 fold for CD34⁺ and CD14⁺ cells, and almost 5 fold for CD19⁺ B lymphoid cells.

The increased transgene expression level was also observed in both in vivo human HSC repopulation models. Addition of SAR to either retroviral backbone increased the MFI of transgene expression 2.5 to 4 fold in vivo. This increased level of expression has been shown to be true for multiple hematopoietic lineages: CD19⁺, CD33⁺, and CD14+cells in the NOD/SCID model, and for human thymocytes in the SCID-hu thy/liver model (Austin et al. manuscript in preparation).

TABLE 1
Comparison of MoMLV-SAR and MSCV1-SAR vectors
for NGFR transgene expression in the NOD/SCID assay

| | | | | Mean % |
|---|---|---|---|---|
| Retroviral | Mouse | Number of mice | Mean % | CD45⁺ |
| Vector | Tissue | with CD45⁺ cells | CD45⁺ cells* | cells, NGFR⁺ |
| MoMLV- | BM | 6/6 | 41-46 | 0.28 ± 0.49 |
| SAR | PB | 4/6 | 21-49 | 1.25 ± 0.45** |
| MSCV1- | BM | 6/6 | 49-82 | 4.30 ± 0.38 |
| SAR | PB | 6/6 | 15-52 | 5.00 ± 0.85 |

Footnote to Table 1
Mobilized CD34⁺ cells from the same donor were transduced with either MoMLV-SAR or MSCV1-SAR vectors.
*mean of 3 mice with human cells detectable in PB.ND = not determined.

*The 2 numbers represent the mean % human cells following i.v. injection of 10 and 20 million cells per mouse, respectively. CD45 stains human hematopoietic cells. The right hand column shows the mean transgene expression among human cells in bone marrow (BM) or peripheral blood (PB) of 6 mice±standard error of the mean (SEM).

**mean of 4 mice with human cells detectable in PB.

TABLE 2A
Comparison of MSCV and MSCV1-SAR vectors for
NGFR transgene expression in the NOD/SCID assay

| | | Number | | | |
|---|---|---|---|---|---|
| | | of Mice | | | Mean % of |
| | | with | | Mean % of | human cells |
| | Mouse | CD45⁺ | Mean % | CD45⁺ cells, | marked with |
| Vector | tissue | cells | CD45⁺ cells | NGFR⁺ | IRES-DHER |
| MSCV1 | BM | 4/4 | 38.3 ± 8.7 | 1.63 ± 0.23 | 1.63 ± 0.3 |
| | PB | 3/4 | 15.4 ± 6.2* | 1.18 ± 0.72* | ND |
| MSCV1- | BM | 4/4 | 41.4 ± 2.1 | 3.63 ± 0.2 | 4.46 ± 0.54 |
| SAR | PB | 3/4 | 14.1 ± 13* | 3.20 ± 0.7* | ND |

Footnote to Table 2A

Mobilized CD34⁺ cells from the same donor were transduced with either MSCV1 or MSCV1-SAR vectors. Ten million cells post transduction were injected i.v. into each NOD/SCID mouse. Data=mean of 4 mice±SEM.

*mean of 3 mice with human cells detectable in PB. ND×not determined.

TABLE 2B
Comparison of MFI of NGFR expression and proportion of human cells
with high level transgene expression in NOD/SCID mouse bone marrow

| | | % of total human | MFI of NGFR |
|---|---|---|---|
| Retroviral | % NGFR⁺ of | cells with high | expression |
| Vector | total human cells | NGFR fluorescence | (total cells) |
| MSCV1 | 1.63 ± 0.23 | 13.4 ± 2.4 | 245 |
| MSCV1-SAR | 3.63 ± 0.2 | 56.1 ± 2.5 | 860 |
| | | % of CD19⁺ cells | MFI of NGFR |
| Retroviral | % NGFR⁺ | with high NGFR | expression |
| Vector | of CD19⁺ | fluorescence | (CD19⁺ cells) |
| MSCV1 | 1.3 ± 0.1 | 19.7 ± 2.2 | 347 |
| MSCV1-SAR | 4.4 ± 0.4 | 60.9 ± 3.2 | 990 |
| | | % of CD33⁺ cells | MFI of NGFR |
| Retroviral | % NGFR⁺ | with high NGFR | expression |
| Vector | of CD33⁺ | fluorescence | (CD33⁺ cells) |
| MSCV1 | 1.1 ± 0.4 | 4.0 ± 2.3 | 201 |
| MSCV1-SAR | 3.8 ± 1.5 | 29.4 ± 4.5 | 501 |

Footnote to Table 2B
The gate setting to determine the percentage of cells with high transgene expression (>10³ fluorescence units) out of NGFR⁺ human cells is shown in FIG. 5. MFI is the mean fluorescence intensity. CD19 expression identifies human B lymphoid cells and CD33 expression identifies human myeloid cells.

### EXAMPLE 2

Our current clinical trial uses a MoMLV vector containing RevM10 anti-HIV transgene (Malim et al., 1989). There appears to be a threshold level for the RevM10 protein to allow efficient competition with the normal HIV Rev protein (Plavec et al., 1992). It is now investigated whether the addition of SAR to a MSCV retroviral vector increases the level of in vivo RevM10 production per cell. Indeed, compared with a standard retroviral vector, a MoMLV-SAR vector was significantly more potent for inhibition of HIV-1 replication in CD4⁺ PBL (Auten et al., 1999). Use of an optimized transduction protocol and an improved MSCV1-SAR vector has an important therapeutic value for inhibition of HIV replication in vivo, as well as for production of therapeutic levels of protein in other gene therapy applications.

### EXAMPLE 3

MSCV1-SAR vector of example 1 was used to express the Herpes simplex thymidine kinase gene which renders cells sensitive to antiviral compounds, such as acyclovir, gancyclovir and FIAU (1-(2-deoxy-2-fluoro-.beta.-D-arabinofuranosil)-5-iodouracil).

What is claimed is:

1. A retroviral vector comprising:
(a) at least one transgene operatively linked to a promoter derived from MSCV; and
(b) a DNA scaffold attachment region (SAR element).

2. The retroviral vector of claim 1, wherein the promoter further comprises the MESV promoter, MND promoter, SFFVp promoter or FMEV promoter.

3. The retroviral vector of claim 1, wherein the SAR element inhibits methylation of the 5′ LTR of the retroviral vector.

4. The retroviral vector of claim 3, wherein the SAR element is HIFN-β SAR.

5. A retroviral vector of claim 1, wherein the transgene is RevM10 or an antisense of the HIV reverse polymerase.

6. A method of increasing expression of a transgene in a retrovirally transduced eukaryotic resting cell, comprising:
(a) transducing a eukaryotic cell with a retroviral vector, the retroviral vector comprising (i) a transgene operatively linked a promoter derived from MSCV, and (ii) a scaffold attachment region (SAR); and
(b) expressing the transgene.

7. The method of claim 6, wherein wherein the promoter further comprises the MESV promoter, MND promoter, SFFVp promoter or FMEV promoter.

8. A retrovirus particle comprising the retroviral vector of claim 1.

9. A retrovirus particle comprising the retroviral vector of claim 2.

10. A retrovirus particle comprising the retroviral vector of claim 3.

11. A retrovirus particle comprising the retroviral vector of claim 4.

12. A retrovirus particle comprising the retroviral vector of claim 5.

13. A cell line comprising the retrovirus particle of claim 8.

14. A cell line comprising the retrovirus particle of claim 9.

15. A cell line comprising the retrovirus particle of claim 10.

16. A cell line comprising the retrovirus particle of claim 11.

17. A cell line comprising the retrovirus particle of claim 12.
